(19) United States
(12) Patent Application Publication
(10) Pub. No.: US 2011/0245817 A1
(43) Pub. Date: Oct. 6, 2011

(54) OPHTHALMIC LASER TREATMENT APPARATUS

(75) Inventors: Hiroki YOKOSUKA, Gamagori-shi (JP); Koshu TAJITSU, Nukata-gun (JP); Shinichi MATSUURA, Toyokawa-shi (JP)

(73) Assignee: NIDEK CO., LTD., Gamagori-shi (JP)

(21) Appl. No.: 13/073,278

(22) Filed: Mar. 28, 2011

(30) Foreign Application Priority Data
Mar. 31, 2010 (JP) 2010-084688

(51) Int. Cl. A61B 18/20

(52) U.S. Cl. 606/4

(57) ABSTRACT

An ophthalmic laser treatment apparatus for treating a patient's eye, including: a binocular microscopic optical system; a treatment laser source; an optical fiber; an irradiation optical system, the irradiation optical system including: a zoom optical system including a zoom lens movable along an optical axis of the irradiation optical system; a scanner; an aperture plate placed on an optical path between the zoom optical system and the scanner, the aperture plate including an aperture; an image forming optical system including an image forming lens; and a reflection mirror placed at a center between right and left optical paths of the binocular microscopic optical system; a controller for controlling driving of the scanner based on an irradiation pattern in which a plurality of the irradiation spots of the treatment beam are arranged.

### CROSS-REFERENCE TO RELATED APPLICATIONS

This application is based upon and claims the benefit of priority from the prior Japanese Patent Application No. 2010-084688, filed Mar. 31, 2010, the entire contents of which are incorporated herein by reference.

### TECHNICAL FIELD

The present invention relates to an ophthalmic laser treatment apparatus for treating a patient's eye by irradiation a laser beam thereto.

### BACKGROUND ART

As one of ophthalmic laser treatment apparatuses, a photocoagulation apparatus is known. For photocoagulation treatment (e.g., panretinal photocoagulation treatment), a treatment laser beam is sequentially irradiated on a spot-by-spot basis to fundus tissues of a patient's eye to thermally photocoagulate the tissues (for example, see JP 2002-224154A). In recent years, an apparatus has been proposed in which a scanning unit including a galvano mirror and others is installed in a laser-beam delivery unit to scan a treatment laser beam in the form of a spot onto fundus tissues based on a plurality of irradiation patterns of spot positions set in advance (for example, WO07/082,102). This apparatus is configured to change a spot size (a spot diameter) of the laser beam according to treatments. For instance, an apparatus in JP 2002-224154A uses a zoom optical system including lenses movable in an optical axis direction of the laser beam to change a spot size. On the other hand, the apparatus in WO07/082,102 is provided with a plurality of optical fibers having different core diameters to deliver a laser beam emerging from an emission end of each optical fiber onto the tissues. At that time, the optical fibers with different core diameters are selectively used to change the spot size. This apparatus also uses a slit lamp provided with a unit, e.g. a binocular microscope, including an observation optical system for observing the fundus tissues of the patient's eye and checking the irradiation positions of spots.

### SUMMARY OF INVENTION

### Technical Problem

The apparatus of WO07/082,102 has limited spot size variations. Therefore, such an apparatus as disclosed in JP 2002-224154A arranged to scan the spot formed through the zoom optical system is demanded. However, if a scanner is installed in the zoom optical system, a problem occurs in which an irradiation range of the spots is limited.

To be concrete, a conventional apparatus (such as the apparatus in JP 2002-224154A) is shown in FIGS. 7A and 7B; FIG. 7A is a side view and FIG. 7B is a schematic top view. A reflection mirror 103 that reflects visible light is placed to make an optical axis La of a laser beam passing through an objective lens 101 of a zoom optical system (an illumination optical system) Z1 almost coincident or coaxial with an observation optical path (an optical axis) Lb of an objective lens 102 of a binocular microscope M1. The reflection mirror 103 is of such a size as not to obstruct a right-eye observation optical path Lbr and a left-eye observation optical path Lb1 of the microscope M1 and is placed at a center between the right-eye and left-eye observation optical paths. Due to these conditions, the size of the reflection mirror 103 is limited. Further, between the objective lens 101 and the reflection mirror 103, an aperture plate 104 is placed to prevent a laser beam from missing the reflection mirror 103 and scattering in front of a patient's eye and others. In the case where the magnification of a spot changed by the zoom optical system Z1 is low, e.g., 50 μm which is the same size as a fiber end face, the aperture plate 104 blocks a resultant beam Ba.

Under those conditions, as shown in FIG. 8, if a scanner 115 such as a galvano mirror is placed in a zoom optical system Z2 and further an objective lens 111, a reflection mirror 113, and an aperture plate 114 are arranged, an optical axis La1 of a laser beam deflected by the scanner 115 is blocked by the aperture plate 114. Thus, a spot irradiation range is limited.

The present invention has been made to solve the above problems and has a purpose to provide an ophthalmic laser treatment apparatus capable of ensuring freedom of choice of spot size and providing a wide spot irradiation range.

### Solution to Problem

To achieve the above purpose, one aspect of the invention provides an ophthalmic laser treatment apparatus for treating a patient's eye, comprising: a binocular microscopic optical system for observing the patient's eye; a treatment laser source for emitting a treatment laser beam; an optical fiber for delivering the treatment beam from the laser source; an irradiation optical system for irradiating the treatment beam emitted from the optical fiber to the patient's eye, the irradiation optical system comprising: a zoom optical system including a zoom lens movable along an optical axis of the irradiation optical system, the zoom optical system being arranged to change a size of an irradiation spot of the treatment beam to be irradiated to the patient's eye; a scanner for scanning the irradiation spot of the treatment beam in two dimensions on tissues of the patient's eye; an aperture plate placed on an optical path between the zoom optical system and the scanner, the aperture plate including an aperture to restrict a sectional diameter of the treatment beam having passed through the zoom lens; an image forming optical system including an image forming lens for focusing the treatment beam having passed through the aperture and being emitted from the scanner on the tissues; and a reflection mirror placed at a center between right and left optical paths of the binocular microscopic optical system, the reflection mirror being arranged to reflect the treatment beam having passed through at least a part of the image forming lens toward the patient's eye; a controller for controlling driving of the scanner based on an irradiation pattern in which a plurality of the irradiation spots of the treatment beam are arranged, and the aperture has a size to restrict the treatment beam from missing the reflection mirror when the size of the irradiation spot of the treatment beam is changed by the zoom optical system to a predetermined low magnification value or less, the scanner is not operated, and a center of the treatment beam is made coincident with an optical axis of the image forming optical system.

### BRIEF DESCRIPTION OF DRAWINGS

FIG. 1 is a schematic configuration view of optical systems and a control system of an ophthalmic laser treatment apparatus;

FIG. 2 is a perspective view of a scanner;

FIG. 3 is a schematic optical view to explain a laser irradiation optical system;

FIGS. 4A and 4B are explanatory views showing a relationship between a reflection mirror and a spot size;

FIG. 5 is a view showing one example of irradiation patterns;

FIG. 6 is a graph showing a relationship between the spot size and an irradiation range;

FIGS. 7A and 7B are explanatory views showing delivery of a laser beam in a conventional ophthalmic laser treatment apparatus; and

FIG. 8 is an explanatory view showing delivery of a laser beam in an ophthalmic laser treatment apparatus including a scanner.

### DESCRIPTION OF EMBODIMENTS

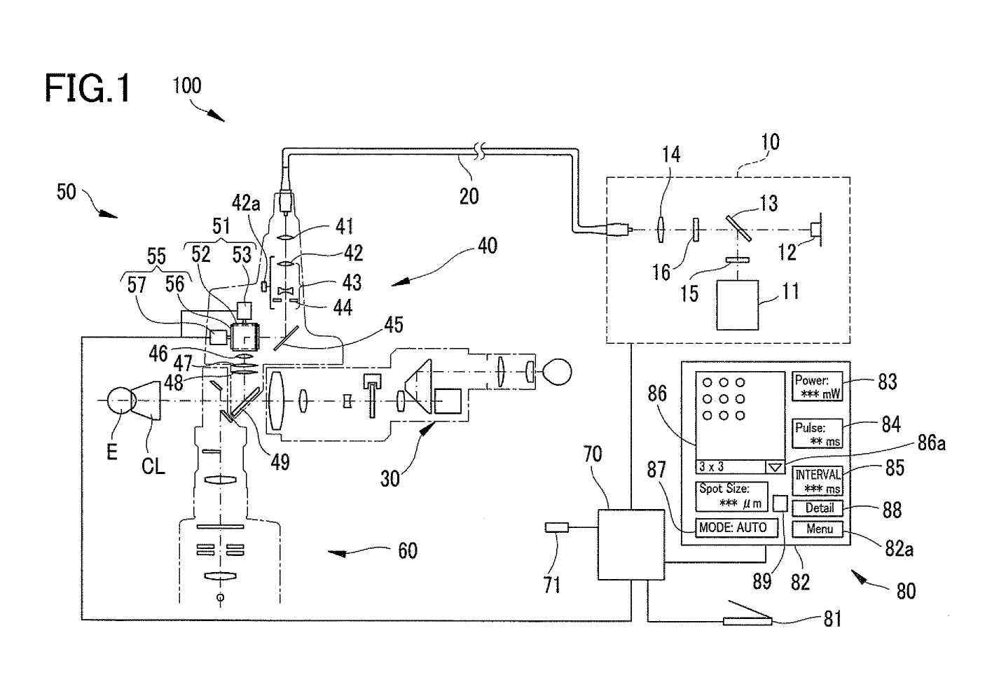

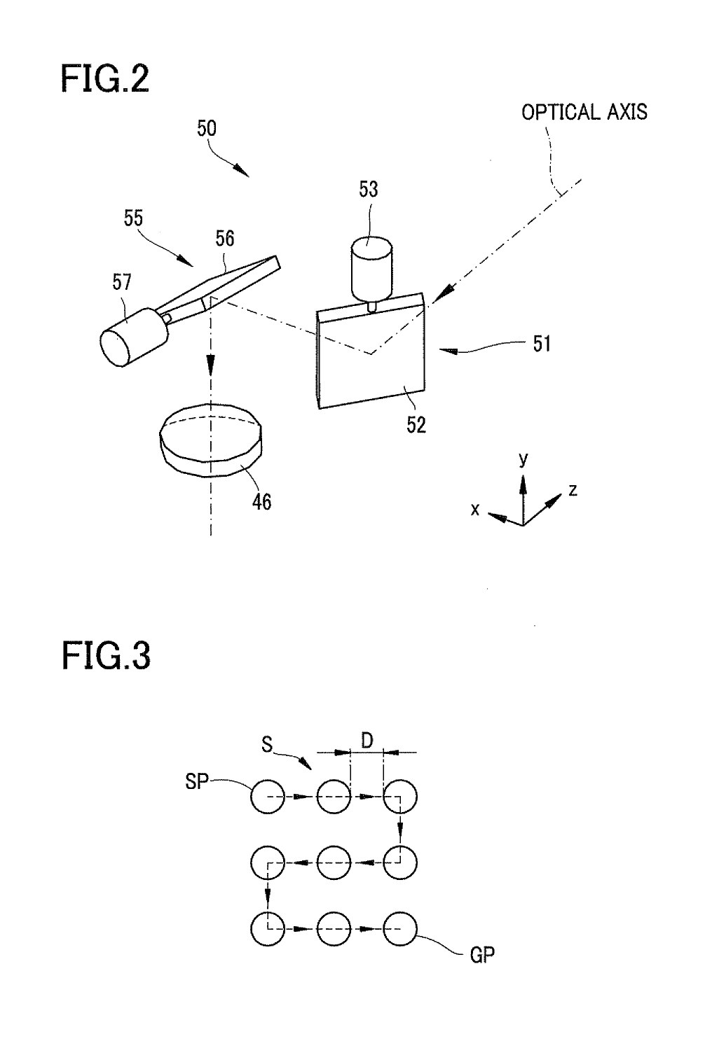

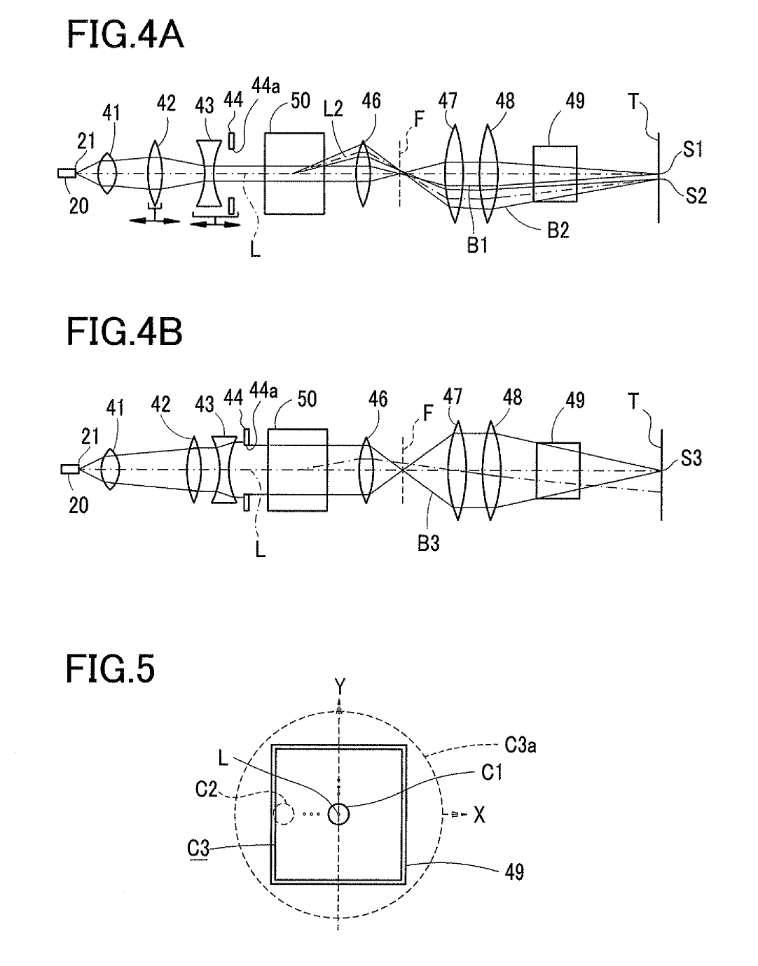

A detailed description of a preferred embodiment of the present invention will now be given referring to the accompanying drawings. FIG. 1 is a schematic configuration view showing optical systems and a control system in an ophthalmic laser treatment apparatus for performing photocoagulation treatment of fundus, and others. FIG. 2 is a perspective view of a scanner. FIG. 3 is a schematic optical view to explain a laser irradiation optical system. FIGS. 4A and 413 are explanatory views showing a relationship between a reflection mirror and a spot size. FIG. 5 is a view s showing one example of irradiation patterns.

An ophthalmic laser treatment apparatus 100 roughly includes a laser source unit 10, a laser irradiation optical system 40, an observation optical system (a binocular microscopic optical system) 30, an illumination optical system 60, a controller 70, and an operation unit 80. The laser source unit 10 includes a treatment laser source 11 for emitting a treatment laser beam, an aiming light source 12 for emitting a visible aiming laser beam (an aiming beam), a beam splitter (a combiner) 13 for combining the treatment laser beam and the aiming beam, and a focusing lens 14. The beam splitter 13 reflects most of the treatment laser beam and transmits a part of the aiming beam. The combined laser beam is focused by the focusing lens 14 to enter an incident end face of an optical fiber 20 for delivering the laser beam to the laser irradiation optical system 40. A first shutter 15 is placed between the laser source 11 and the beam splitter 13 to block the treatment laser beam. Further, a second shutter 16 is placed on an optical path of the aiming beam from the aiming light source 12 and the treatment laser beam from the treatment laser source 11. The second shutter 16 is a safety shutter that is closed in case an abnormality occurs, but also may be used for enabling or blocking of irradiation of the aiming beam during scanning of the aiming beam. The first shutter 15 also may be used for enabling or blocking of irradiation of the treatment laser beam. Each shutter may be replaced with a galvano mirror having a function of switching optical paths. As the optical fiber 20, a multi-mode fiber with a core diameter of 50 μm and NA (numerical aperture) of 0.1 is used.

The laser irradiation optical system 40 is configured as a delivery unit mounted in a slit lamp (not shown) in the present embodiment. A laser beam (the treatment laser beam and the aiming beam) emitted from the optical fiber 20 is delivered by the following optical elements. Specifically, the laser beam passes through a collimator lens 41, zoom lenses 42 and 43 movable in an optical axis direction to change a spot size of the laser beam, an aperture plate 44 having an aperture configured to restrict a beam diameter, and a mirror 45 for deflecting an optical path. The beam deflected by the mirror 45 passes through a scanner (a scanner) 50, an image forming lens 46, a relay lens 47, an objective lens 48, and a reflection mirror 49 and is irradiated onto a fundus of a patient's eye E. The reflection mirror 49 is placed at a center between right and left optical paths of the observation optical system 30.)

The scanner 50 is a unit constituting a scanning optical system including a scanner mirror for moving an irradiation direction (an irradiation position) of the laser beam in two dimensions. The scanner 50 includes a first galvano mirror (a galvano scanner) 51 and a second galvano mirror 55. The first galvano mirror 51 includes a first mirror 52 for reflecting the laser beam and an actuator 53 serving as a drive part for driving (rotating) the mirror 52. Similarly, the second galvano mirror 55 includes a second mirror 56 and an actuator 57. The laser beam having passed through each optical element of the laser irradiation optical system 40 is reflected by the reflection mirror 49 and irradiated onto the fundus which is a target surface (onto the tissues) of the eye E through a contact lens CL.

The zoom lenses 42 and 43 constituting a zoom lens group are held in a lens cam not shown. The lens cam is rotated by operation of an operator to move each zoom lens 42 and 43 in an optical axis direction. The positions of the zoom lenses 42 and 43 are detected by an encoder 42a attached to the lens cam. The controller 70 for integrally controlling the apparatus 100 receives positional information (a detection signal) of each lens from the encoder 42a and obtains a spot size of the laser beam. This provides a spot size input device. The spot size may be inputted on a display 82 by an operator. The encoder 42a serves as an input switch for the spot size.

The scanner 50 is controlled based on a command signal from the controller 70 to scan spot positions to form an irradiation spot (hereinafter, a “spot”) of the set laser beam in a two-dimensional irradiation pattern on the target surface. The reflection mirror 49 is connected to a mechanism (a hand-operated manipulator), not shown, which is operated by the operator, to tilt the optical axis of the laser beam in two dimensions.

The structure of the scanner 50 will be explained. As shown in FIG. 2, the mirror 52 is attached to the actuator 53 to swing a reflection plane in an x-direction. On the other hand, the mirror 56 is attached to the actuator 57 to swing a reflection plane in a y-direction. In the present embodiment, the rotation axis of the mirror 52 coincides with a y-axis and the rotation axis of the mirror 56 coincides with a z-axis. Further, the actuators 53 and 57 are connected to and separately driven by the controller 70. Each of the actuators 53 and 57 contains a motor and a potentiometer (both not shown). The mirrors 52 and 56 are independently rotated (swung) based on command signals from the controller 70. At that time, positional information representing how much the mirrors 52 and 56 have been rotated is transmitted from the potentiometers of the actuators 53 and 57 to the controller 70. Accordingly, the controller 70 ascertains the rotational positions of the mirrors 52 and 56 with respect to the command signals.

The observation optical system 30 and the illumination optical system 60 are installed in the slit lamp. The observation optical system 30 includes an objective lens and further a variable magnification optical system, a protection filter, erect prisms, a field diaphragm, eyepieces, and others. The illumination optical system 60 for illuminating the eye E with slit light includes an illumination light source, a condenser lens, a slit, a projection lens, and others.

To the controller 70, there are connected a memory 71, the light sources 11 and 12, the encoder 42a, the actuators 53 and 57, the operation unit 80, and a footswitch 81 serving as a device for inputting a trigger for irradiation of the laser beam. The operation unit 80 includes a touch panel display 82 used for setting laser irradiation conditions, and also changing and inputting irradiation patterns. The display 82 is provided with various panel switches for setting parameters of the laser irradiation conditions. The display 82 has a graphical user interface function enabling a user to visually check and set numerical values and others. For items of the irradiation conditions, there are prepared a setting part 83 for output power of the treatment laser beam, a setting part 84 for an irradiation time (a pulse width), a setting part 85 for a halt time (a time interval of irradiation of the treatment laser beam), a setting part 86 for irradiation patterns of the treatment laser beam (arrangement patterns of spot positions of the treatment laser beam to be formed on the target plane), a mode setting part 87 for setting an aiming mode, a details setting switch 88, a spot interval setting device (a spot interval setting part) 89, a menu switch 82a for calling up other setting parts and others, etc. With the mode setting part 87, a plurality of aiming modes is selectively set.

At the touch of each item on the display 82, numeral values can be set. For instance, when an operator touches the switch 86a, selectable options are displayed in a pull-down menu. When the operator chooses a numeral value from the options, a set value in that item is determined.

A plurality of irradiation patterns is previously prepared to be selectable by the operator on the display 82. As the irradiation patterns prepared by an apparatus manufacturer, for example, there are a pattern of spots arranged in a square matrix of 2×2, 3×3, 4×4 or other (a square pattern), a pattern of spots arranged in a circular arc form (a circular arc pattern), a pattern of spots arranged in an outer circumferential direction and an inner circumferential direction to form a fan-like form (a fan-like pattern), a pattern of spots arranged in a circular form (a circular pattern), a segmental pattern of the circular pattern (a circular segmental pattern), a linear pattern of spots arranged in a linear form, and other patterns. They are stored in the memory 71. The irradiation pattern is selectable from the plurality of irradiation patterns stored in the memory 71 by use of the switch 86a on the setting part 86. A selected irradiation pattern is displayed on the screen of the setting part 86. Further, the information of the size of the irradiation size of the laser beam changed by movement of the zoom lenses 42 and 43 is displayed on the display 82.

Further, the memory 71 stores irradiation range information for setting a spot irradiation (scanning) range based on a set spot size, a selected irradiation pattern, and a set spot interval. The irradiation range information will be mentioned later.

When the footswitch 81 is pressed down by the operator, the controller 70 irradiates the laser beam based on the settings of various parameters to form a pattern of the treatment laser beam on the target surface. Specifically, the controller 70 controls the light source 11 and controls the scanner 50 based on the set pattern to form the pattern of the treatment laser beam on the target surface (the fundus).

The controller 70 inhibits the scanner 50 from scanning when the set spot size is a predetermined low magnification (power) or less. To be concrete, the controller 70 disables the selection of irradiation pattern on the display 82 (the switch 86a). The controller 70 also sets the optical axis of the scanner 50 as an original position.

FIG. 3 shows one example of the patterns of spot positions. As shown in FIG. 3, this pattern is configured by arranging spots S in a 3×3 square matrix. Herein, the spots S represent both the aiming beam and the treatment laser beam. Based on this pattern, the treatment laser beam and the aiming beam are scanned by the scanner 5 to form the pattern on a target surface. The spot S starts to be irradiated from a start position SP and is scanned toward an end position GP in two dimensions. In the present embodiment, as indicated by an arrow in the figure, the laser beam is scanned to sequentially move from one to adjacent spots S so as to enable movement between spots S as efficient as possible.

An interval D between the spots S can be arbitrarily set in a range from 0.5 to 2 times the spot diameter by a spot interval setting part 89. Setting information of the spot interval D is inputted into the controller 70. In the case of the square pattern shown in FIG. 3, the interval D is determined so that the spots S are arranged at equal intervals in vertical and horizontal directions.

A structure of a zoom optical system in a laser irradiation optical system will be explained below. In FIGS. 4A and 4B, the optical elements are schematically arranged linearly between the fiber emission end 21 and a target surface T. In FIG. 5, a beam in section at the position of the reflection mirror 49 is illustrated as a circle for easy explanation.

The zoom optical system in this embodiment is configured as a parfocal optical system for enlarging the laser beam emitted from the end face of the fiber emission end 21 to a spot with a predetermined spot size and then forming an image of the spot on the target surface T. The beam emerging from the fiber emission end 21 is collimated into parallel light (herein, slightly dispersed light) having a first sectional diameter by the collimator lens 41 which is a convex lens. The zoom lenses 42 and 43 serve to change a beam diameter and deliver the beam having passed through the lens 43 in the form of parallel light having a second sectional diameter to the scanner 50. The zoom lens 42 is a convex lens and the zoom lens 43 is a concave lens. Both lenses 42 and 43 are moved in conjunction with each other along an optical axis L. Herein, the zoom lens 42 acts as a variator and the zoom lens 43 acts as a compensator. When the zoom lenses 42 and 43 are moved continuously, the spot size is changed consecutively. In this embodiment, the spot size is set to 50 to 500 μm (1× to 10× magnification). An aperture plate 44 is fixed downstream of the zoom lens 43. This aperture plate 44 has an aperture 44a shaped to restrict the sectional diameter of the laser beam to be delivered to the scanner 50 when the spot size is a certain value or less (within a low magnification range).

The spot size within the low magnification range represents a range in which a beam diameter in the position of the reflection mirror 49 is larger than a reflection surface of the reflection mirror 49 when the laser beam with a spot size set to a certain value is to be delivered onto the target surface T. In other words, it indicates a magnification range whereby causing the beam to miss or fall outside the reflection mirror 49 without being reflected by the reflection mirror 49. Herein, a spot in the low magnification range is referred to as a small spot size and a spot with a larger spot size than that small spot size is referred to as a large spot size.

In the present embodiment, concretely, the low magnification range corresponds to a spot size of 50 μm or more and less than 100 μm. In this embodiment, the aperture plate 44 having the aperture 44a to restrict a light beam with a spot size of 50 μm to 99 μm (1 to about 2 times the diameter of the fiber emission end 21) from exceeding the size of the reflection mirror 49. The aperture 44a is formed in a rectangular shape similar to the reflection mirror 49. In the case where such a small spot size is set, the controller inhibits the scanner 50 from scanning the spot. When the scanner 50 is not operated to scan and the center (the optical axis) of the treatment laser beam is made coincident with the optical axis of the image forming lens 46 and the objective lens 48, the aperture plate 44 restricts the light beam to prevent the treatment laser beam from missing or falling outside the reflection mirror 49.

The scanner 50 is placed downstream of the aperture plate 44. For facilitating explanation, the scanner 50 is shown only to deflect a laser beam in the X direction. Downstream of the scanner 50, an image forming lens group (the image forming lens 46 to the objective lens 48) is disposed. The light beam having passed through the image forming lens 46 focuses to form an intermediate image in front of the relay lens 47, i.e., in an image forming position F, before reflection by the reflection mirror 49. The relay lens 47 and the objective lens 48 form an image of the spot in the image forming position F onto the target surface T through the reflection mirror 49. Since the intermediate image is formed in a position near and downstream of the scanner 50, the optical elements placed behind the image forming position F can have a smaller diameter.

A relationship between the spot size and the aperture plate 44 is explained below. FIG. 4A shows a case where a large spot size (e.g., 500 μm) is set. FIG. 4B shows a case where a small spot size (e.g., 50 μm) is set. In FIG. 4A, there are shown an on-axis beam B1 corresponding to the optical axis L when the scanner 50 is not operated (the optical axis of the scanner 50 is in the original position) and a beam B2 corresponding to an optical axis L2 when the scanner 50 is operated to deflect the optical axis L to the optical axis L2. The beam B1 is changed in beam diameter and collimated into parallel light by the zoom lenses 42 and 43. This parallel light passes through the scanner 50 and the lenses 46 to 48 and then is focused onto the target surface T, forming a spot S1 thereon. The beam B2 is delivered in a similar way to the beam B1, and deflected to the optical axis L2 by the scanner 50, forming a spot S2 in a peripheral position on the target surface T. At that time, the beams B1 and B2 in the position of the reflection mirror 49 (i.e., on the reflection surface) are schematically illustrated in cross sections C1 and C2 in FIG. 5.

In FIG. 4B, on the other hand, an on-axis beam B3 corresponding to the optical axis L is delivered onto the target T without being deflected by the scanner 50, thus forming a spot S3. At that time, the beam B3 in the position of the reflection mirror 49 is illustrated in a cross section C3. A beam in the case where the beam B3 is not restricted by the aperture plate 44 is illustrated in a cross section C3a.

In the case where the small spot size is set, the zoom lenses 42 and 43 are moved to maximize the beam diameter in the position of the aperture plate 44. This depends on characteristics of the zoom optical system in the parfocal optical system and is determined based on a relationship among NA of the fiber 20, the magnification (1×) of the spot size, and the optical elements of the zoom optical system. The aperture plate 44 restricts the beam diameter of the beam B3 and guides the beam to the scanner 50. This beam B3 is reflected as the cross section C3 by the reflection mirror 49. If the aperture plate 44 is absent, the beam B3 will have a cross section C3a in the position of the reflection mirror 49. In this case, the light falling outside of the reflection mirror 49 is not reflected by and misses the reflection mirror 49. Therefore, the aperture 44a of the aperture plate 44 is designed to have a sectional area enough to restrict the beam diameter corresponding to the small spot size and provide the cross section C3 as large as possible on the reflection surface of the reflection mirror 49.

The beam B3 has a diameter (a dimension) ensuring as wide a diameter as possible on the reflection mirror 49 and thus could not be scanned by the scanner 50. Further, a beam corresponding to the small spot size is not suitable for scanning by the scanner 50. The controller 70 therefore disables the scanner 50 from scanning when the small spot size (a corresponding value) is set based on the input of the encoder 42a.

On the other hand, when the large spot size is set, both the beams B1 and B2 are not restricted in beam diameter by the aperture plate 44 as shown in FIG. 4A. As shown in the cross sections C1 and C2 on the reflection mirror 49, the beams B1 and B2 are smaller than the area of the reflection mirror 49. Thus, the beam corresponding to the large spot size does not miss the reflection mirror 49 even when the optical axis is deflected by the scanner 50. For instance, even when the beam B2 is deflected as the optical axis L2 from the beam B1 on the optical axis L, the beam B2 converge as the cross section C2 on the reflection mirror 49.

However, the reflection mirror 49 is limited in size as mentioned above. Even when the large spot size is set, accordingly, a spot scanning range is limited. The controller 70 performs a comparative calculation of the set spot size, the irradiation pattern, and the spot interval with the irradiation range information previously stored in the memory 71 to set a range to be scanned by the scanner 50 and also restrict the irradiation range in the current setting.

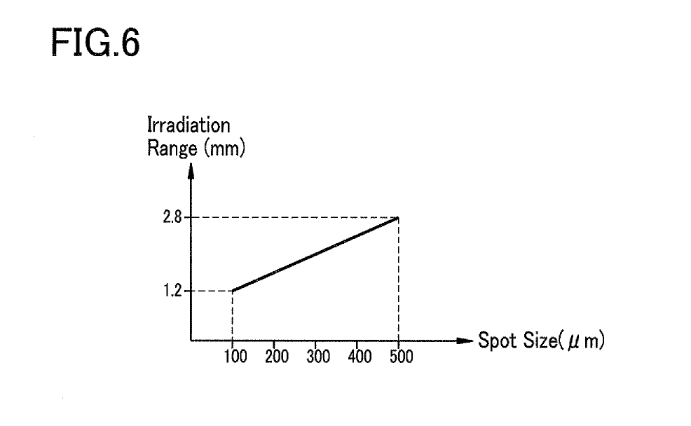

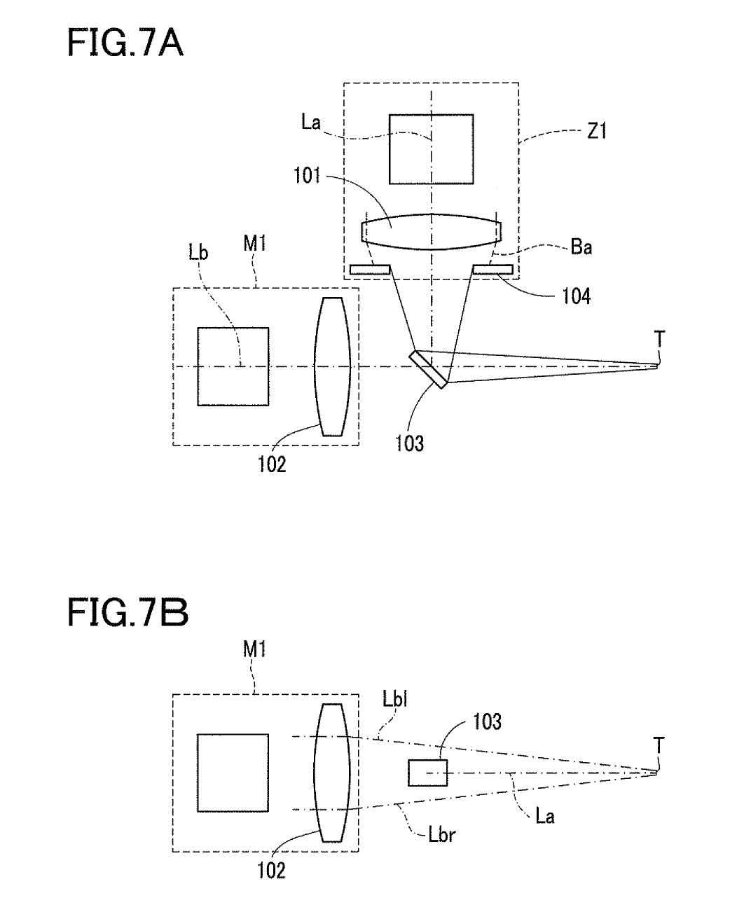

FIG. 6 is a graph showing a relationship between the spot size and the irradiation range. A lateral axis represents the spot size and a vertical axis represents the irradiation range in one-side direction on the target surface. As seen in the graph, the scanning is not enabled as long as the spot size is less than 100 μm and thus the irradiation range is zero. For 100 μm or more, the irradiation range is wider as the spot size is larger. This is because the beam in the position of the reflection mirror 49 becomes narrower as the spot size increases, so that the beam can be scanned on the reflection mirror 49, i.e., swung in the X and Y directions.

Data shown in this graph is stored as the irradiation range information in the memory 71 and used for setting an irradiation range by the controller 70. For instance, when the spot size is 500 μm, the spot is allowed to scan a range of 2.8 mm in the X and Y directions. The irradiation pattern and the spot interval are determined so as to fall within this irradiation range. Based on this limitation, the controller 70 restricts selection and display of parameters on the display 82. Accordingly, the scanning can be performed without causing optical vignetting (mechanical vignetting) in the optical elements such as the reflection mirror 49 and others.

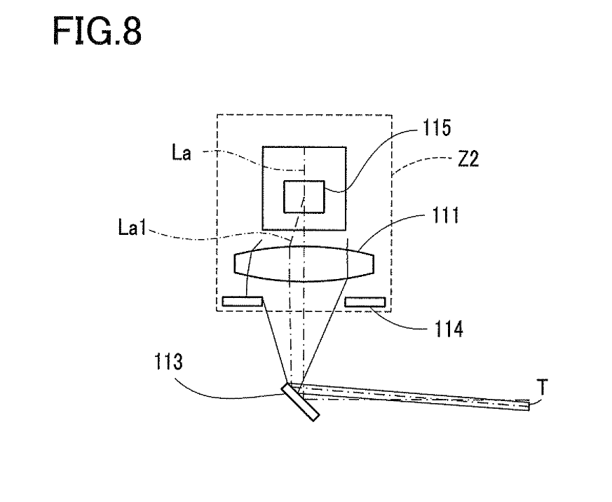

As above, the controller 70 sets as large a range as possible of the spots to be formed by the irradiation optical system 40. In the irradiation optical system 40, the aperture plate 44 is not disposed on an optical path from the scanner 50 to the reflection mirror 49 and is disposed downstream of the zoom lens group, i.e., at a scanner side position than a position of the zoom lens group, to restrict the cross-sectional diameter of the beam before entering the scanner 50. This configuration can provide the following advantages. Firstly, when the small spot size is set, the beam diameter can be restricted, thereby preventing the treatment laser beam and others from missing the reflection mirror 49. Secondly, the treatment laser beam can be delivered to the reflection mirror 49 along an optical path passing through peripheral portions of the image forming lenses. Accordingly, the beam can be delivered onto the target surface T without being blocked as shown in FIG. 8. Thus, the spot irradiation range on the target surface T can be ensured as wide as possible. Thirdly, the number of selectable spot sizes can be increased by the zoom optical system, thus offering improved degree of freedom of spot size, that is, treatment.

The case where the beam passing through the zoom lens 43 becomes parallel light represents the on-axis beam emitted from the fiber emission end 21. Accordingly, an out-of-axis beam emitted from the fiber emission end 21 is not parallel light and becomes slightly diffused light. It is therefore preferable that the aperture plate 44 placed between the zoom lens 43 and the scanner 50 is disposed in a position as near as possible to the zoom lens 43 so as not to block the diffused light of the beam passing through the zoom lens 43 in the case of the large spot size. In this embodiment, the aperture plate 44 is fixed downstream of the zoom lens 43. Specifically, the aperture plate 44 is positioned and secured in a lens holder not shown of the zoom lens 43 with a fixing member such as a screw. The position of the aperture plate 44 is therefore always constant with respect to the zoom lens 43. This configuration can facilitate designing of the aperture plate 44, reduce a space between the zoom lens 43 and the scanner 50, thereby making the irradiation optical system 40 compact.

Operations of the apparatus having the above configuration will be explained below. Prior to a surgical operation, conditions for the operation are set such as irradiation pattern, spot size of the treatment laser beam, output power of the treatment laser beam, and irradiation time of the laser beam in one spot. For example, for panretinal photocoagulation treatment, it is assumed that a spot size of the treatment laser beam is set to 200 μm and a 5×5 square pattern is selected as the irradiation pattern, respectively. At that time, the controller 70 performs comparative calculation of the set spot size, irradiation pattern, and spot interval with the irradiation range information and sets an irradiation range suitable for the parameter and others for the current surgical operation. For instance, the operator sets the spot size and the spot pattern.

The operator observes, through the observation optical system 30, the fundus illuminated by illumination light from the illumination optical system 60 and also the spot positions of the irradiated aiming beam, and moves the slit lamp (consisting of the observation optical system 30 and the illumination optical system 60) containing the laser irradiation optical system 40 relative to the patient's eye E to perform aiming to a treatment area. During the aiming, the driving of the aiming beam 12 and the scanner 50 is controlled based on the irradiation pattern.

After completion of the aiming, when the operator presses the footswitch 81, the irradiation of the treatment laser beam is started. Upon receipt of the trigger signal from the foot switch 81, the controller 70 stops emission of the aiming beam from the laser source 12 and starts emission of the treatment laser beam from the treatment laser source 11, and also controls the scanner 50 to sequentially irradiate the treatment laser beam to each spot position. The treatment laser beam is irradiated to each spot position based on the set time of a pulse width of the treatment laser beam. The spot is moved during the halt time of the treatment laser beam.

In the case where the spot size is set less than 100 μm, the controller 70 disables the scanner 50 from scanning the spots. It may be arranged that a mode of not scanning the spots is referred to as a single mode and a mode of scanning the spots is referred to as a scan mode so that the controller 70 selects either mode based on a set spot size and a set irradiation pattern. Furthermore, for setting the conditions for surgical operation, a configuration may be added to inform an operator of selectable irradiation patterns and conditions for surgical operation when either mode is selected by the operator.

The scanner 50 may include a member for e.g. tilting a single mirror in x and y directions. As an alternative, scanning of the laser beam and others may be conducted by tilting the lens.

In the above explanation, the aperture plate is fixed downstream of the most downstream zoom lens in the zoom lens group. The aperture plate may be fixedly placed upstream of the scanner and downstream of the zoom lenses.

In the above explanation, the shape and the position of the aperture plate are determined so as not to block the out-of-axis beam (the diffused light) of the beam with the spot size larger than the small spot. Alternatively, they may be determined to block the out-of-axis beam. In this case, an amount of energy to be irradiated onto the target surface decreases and therefore an amount of irradiation energy of the treatment is set to be larger.

### REFERENCE SIGNS LIST

10 Laser source unit
20 Optical fiber
21 Fiber emission end
30 Observation optical system
42, 43 Zoom lens
44 Aperture plate
49 Reflection mirror
50 Scanning part
60 Illumination optical system
70 Controller
80 Operation unit
100 Ophthalmic laser treatment apparatus

1. An ophthalmic laser treatment apparatus for treating a patient's eye, comprising:
a binocular microscopic optical system for observing the patient's eye;
a treatment laser source for emitting a treatment laser beam;
an optical fiber for delivering the treatment beam from the laser source;
an irradiation optical system for irradiating the treatment beam emitted from the optical fiber to the patient's eye,
the irradiation optical system comprising:
a zoom optical system including a zoom lens movable along an optical axis of the irradiation optical system, the zoom optical system being arranged to change a size of an irradiation spot of the treatment beam to be irradiated to the patient's eye;
a scanner for scanning the irradiation spot of the treatment beam in two dimensions on tissues of the patient's eye;
an aperture plate placed on an optical path between the zoom optical system and the scanner, the aperture plate including an aperture to restrict a sectional diameter of the treatment beam having passed through the zoom lens;
an image forming optical system including an image forming lens for focusing the treatment beam having passed through the aperture and being emitted from the scanner on the tissues; and
a reflection mirror placed at a center between right and left optical paths of the binocular microscopic optical system, the reflection mirror being arranged to reflect the treatment beam having passed through at least a part of the image forming lens toward the patient's eye;
a controller for controlling driving of the scanner based on an irradiation pattern in which a plurality of the irradiation spots of the treatment beam are arranged, and
the aperture has a size to restrict the treatment beam from missing the reflection mirror when the size of the irradiation spot of the treatment beam is changed by the zoom optical system to a predetermined low magnification value or less, the scanner is not operated, and a center of the treatment beam is made coincident with an optical axis of the image forming optical system.

2. The ophthalmic laser treatment apparatus according to claim 1, further comprising a size setting unit including a switch for setting the size of the irradiation spot to be changed by the zoom optical system,
wherein the controller inhibits the scanner from scanning the treatment beam when the set size by the size setting unit is the predetermined low magnification value or less.

3. The ophthalmic laser treatment apparatus according to claim 2, further comprising:
an interval setting unit including a switch for setting an interval between the irradiation spots in the irradiation pattern; and
a range setting unit for setting an irradiation enabled range of each irradiation spot by the scanner based on the set size by the size setting unit and the set interval by the interval setting unit;
wherein the controller controls driving of the scanner based on the irradiation enabled range set by the range setting unit.

4. The ophthalmic laser treatment apparatus according to claim 1, wherein
the aperture plate is fixedly placed at a scanner side position than a position of the zoom lens.

5. The ophthalmic laser treatment apparatus according to claim 1, wherein
the irradiation optical system includes a collimator lens for collimating the treatment beam emitted from the optical fiber into an almost parallel beam having a first sectional diameter,
the zoom lens includes a variator lens and a compensator lens for collimating the treatment beam having passed through the collimator lens into an almost parallel beam having a second sectional diameter, and
the image forming lens includes an intermediate image forming lens for focusing the treatment beam emitted from the scanner before reflection by the reflection mirror.
